(19) United States
(12) Patent Application Publication
(10) Pub. No.: US 2023/0405253 A1
(43) Pub. Date: Dec. 21, 2023

(54) ENDOTRACHEAL TUBE RETAINER

(71) Applicant: Wichita State University, Wichita, KS (US)

(72) Inventors: Mohammed A. ANSARI, Wichita, KS (US); Joel WHITE, Wichita, KS (US)

(21) Appl. No.: 18/461,215

(22) Filed: Sep. 5, 2023

Related U.S. Application Data
(63) Continuation of application No. PCT/US2022/020972, filed on Mar. 18, 2022.
(60) Provisional application No. 63/162,880, filed on Mar. 18, 2021.

(51) Int. Cl. A61M 16/04

(52) U.S. Cl. CPC A61M 16/0497

(57) ABSTRACT

The present disclosure provides an endotracheal tube retainer for retaining an endotracheal tube. The endotracheal tube retainer has a clip to be supported on a head such that the clip is spaced apart in front of a mouth and is adjustable between an open configuration and a closed configuration. In the closed configuration, the clip defines a tube retention channel that may receive the endotracheal tube such that the clip grips the endotracheal tube to retain it in the clip. The open configuration releases the endotracheal tube such that the endotracheal tube is movable relative to the clip. The clip can be equipped with primary and secondary retention features for holding the clip closed. The clip can be opened and closed by one-handed operation. The retainer has a stabilizer extension used for stabilizing the endotracheal tube during one-handed operation of the clip.

### CROSS-REFERENCE TO RELATED APPLICATION

This application claims priority to U.S. Provisional Patent Application No. 63/162,880, filed Mar. 18, 2021, and entitled Endotracheal Tube Retainer, which is hereby incorporated by reference in its entirety for all purposes.

### FIELD

The present disclosure generally relates to an endotracheal tube retainer, and more specifically, to an endotracheal tube retainer with a clip.

### BACKGROUND

Endotracheal intubation refers to a medical procedure in which an endotracheal tube is placed into the trachea of a patient to open and/or maintain the patient's airway. Endotracheal intubation is performed on patients to prevent respiratory failure (e.g., asphyxiation or airway obstruction). After a patient is intubated, it is often necessary to use a device (e.g., endotracheal tube holder or endotracheal tube retainer) to aid in retaining the position of the endotracheal tube. Endotracheal tube holders help reduce undesired extubation by providing support, stabilization, and securement of endotracheal tubes in intubated patients.

One such example of an endotracheal tube holder is shown in FIG. 1. As shown, the endotracheal tube holder H includes an elongated arching support bar S that stabilizes an endotracheal tube E projecting from a typically infant patient's mouth or nose. Further the endotracheal tube holder H includes a platform P connected to the support bar S, and for being attached to and stabilizing the endotracheal tube E. Typically, the endotracheal tube is secured to the platform P by wrapping tape T about said platform.

### BRIEF SUMMARY

In one embodiment, the present disclosure provides an endotracheal tube retainer for retaining an endotracheal tube in an intubated position on a subject. The endotracheal tube retainer includes a clip configured to be supported on a head of a subject such that the clip is spaced apart in front of a mouth of the subject. Further, the clip is configured to be repeatably adjustable between an open configuration and a closed configuration. When the clip is in the closed configuration, the clip defines a tube retention channel. Further, when the clip is in the closed configuration, the clip can receive the endotracheal tube in the tube retention channel such that the clip grips a longitudinal segment of the endotracheal tube to retain the endotracheal tube in substantially fixed relation with respect to the clip. When the clip is in the open configuration, the clip can release the endotracheal tube such that the endotracheal tube can be movable relative to the clip.

In another embodiment, the present disclosure provides a support bar, a platform, and the endotracheal tube such that the endotracheal tube retainer is supported by the platform. The support bar has a length and a middle portion along the length and can be attached to a head of a subject such that the support bar extends lengthwise in a generally ear-to-ear direction and the middle portion of the support bar is spaced apart in front of the mouth.

Other aspects and features will be apparent hereinafter.

### BRIEF DESCRIPTION OF DRAWINGS

For a better understanding of the nature and objects of the disclosure, reference should be made to the following detailed description taken in conjunction with the accompanying drawings, in which:

FIG. 1 is a perspective of an endotracheal tube holder of the prior art in use.

FIG. 2 is a perspective of an endotracheal tube retainer of the present disclosure in a closed configuration.

FIG. 3 is another perspective of the endotracheal tube retainer in a closed configuration.

FIG. 4 is a perspective of the endotracheal tube retainer in an open configuration.

FIG. 5 is a flow chart illustrating a method of retaining an endotracheal tube using the endotracheal tube retainer.

FIG. 6 is a perspective of another embodiment of an endotracheal tube retainer in an open configuration.

FIG. 7 is another perspective of the endotracheal tube retainer of FIG. 6 in the open configuration.

FIG. 8 is a perspective of the endotracheal tube retainer of FIG. 6 in a closed configuration.

FIG. 9 is a perspective of the endotracheal tube retainer of FIG. 6 in use holding an endotracheal tube.

FIG. 10 is an elevation of the endotracheal tube retainer of FIG. 6 in the open configuration.

FIG. 11 is a cross section taken in the plane of line 11-11 of FIG. 10.

Reference is made in the following detailed description of preferred embodiments to accompanying drawings, which form a part hereof, wherein like numerals may designate like parts throughout that are corresponding and/or analogous. It will be appreciated that the figures have not necessarily been drawn to scale, such as for simplicity and/or clarity of illustration. For example, dimensions of some aspects may be exaggerated relative to others. Further, it is to be understood that other embodiments may be utilized. Furthermore, structural and/or other changes may be made without departing from claimed subject matter. References throughout this specification to “claimed subject matter” refer to subject matter intended to be covered by one or more claims, or any portion thereof, and are not necessarily intended to refer to a complete claim set, to a particular combination of claim sets (e.g., method claims, apparatus claims, etc.), or to a particular claim.

### DETAILED DESCRIPTION

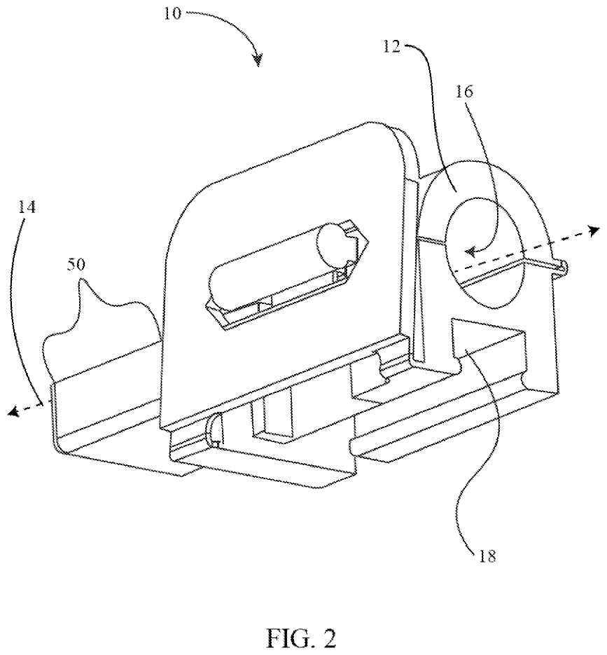

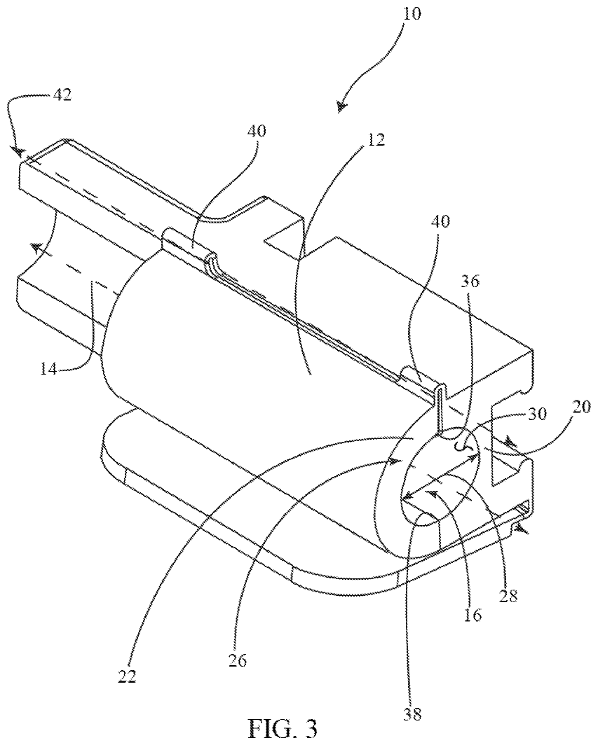

It has been recognized that, after a patient is intubated and the endotracheal tube holder H is secured to the head of the patient, it is often necessary throughout the patient care process to make adjustments to the position of tube. In some instances, adjustments can be made daily or weekly. Using a conventional endotracheal tube holder H, making minor adjustments to the position of the tube sometimes requires removing the tape T, repositioning the tube E, and then re-taping the tube. Biofilm build-up on the tape T, as well as slippage of tape T, often requires re-taping of tape T onto the tube. Referring to FIGS. 2-3, this disclosure provides a new endotracheal tube retainer 10 with a mechanism for securely and repeatably adjusting the position of an endotracheal tube with respect to an endotracheal tube holder.

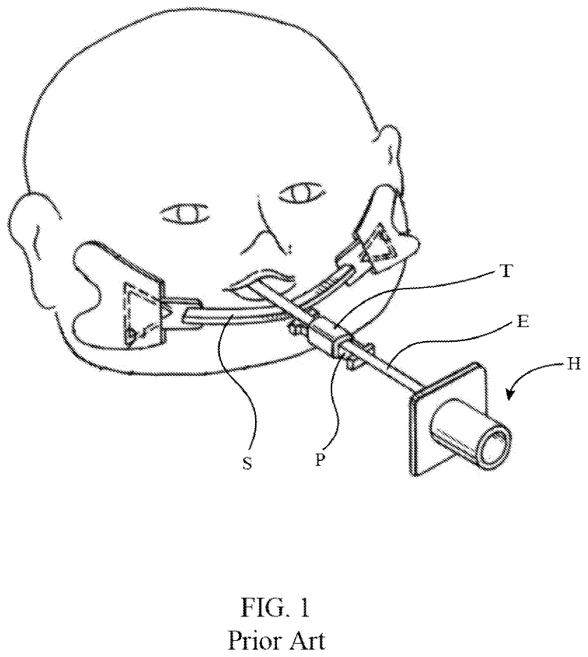

As seen in FIGS. 2-4, the endotracheal tube retainer 10 is broadly configured to retain an endotracheal tube (not shown) in an intubated position on a patient. In general, the endotracheal tube retainer 10 is configured to be used in combination with an endotracheal tube holder of the type depicted in FIG. 1. It is contemplated that the endotracheal tube retainer 10 could be prefabricated as a component of an endotracheal tube holder of the type depicted in FIG. 1 (e.g., the endotracheal tube retainer 10 could be integrally formed with the platform P from a single piece of monolithic material). However, in the illustrated embodiment, the endotracheal tube retainer 10 comprises a separate component that can be selectively attached to the platform P of an existing endotracheal tube holder H shown in FIG. 1. The retainer 10 may be sold separately from the holder H, or the retainer and holder could be packaged together in a kit, optionally, with instructions for installing the retainer on the platform. Suitably, the endotracheal tube retainer 10 is configured to attach to the platform P without tape.

As shown in FIG. 2, the illustrated endotracheal tube retainer 10 of the present disclosure includes a platform coupling 18 for attaching the endotracheal tube retainer to the platform P. The platform coupling 18 includes a snap-on feature that is configured to attach the retainer 10 to the platform P by snap-fit or press-fit connection. For example, in the illustrated embodiment, the platform 18 comprises opposing hook arms that are configured to latch onto a narrow middle portion of the platform P such that the middle portion of the platform is received in the cavity 18, the hook portions of the arms underlie the platform, and the remainder of the retainer 10 is positioned above the platform. Other ways of attaching the endotracheal tube retainer to a separate endotracheal tube holder are also contemplated to be within the scope of the disclosure. For example, the platform coupling 18 may include an adhesive coating that bonds to the platform P when the platform is received in the platform coupling 18.

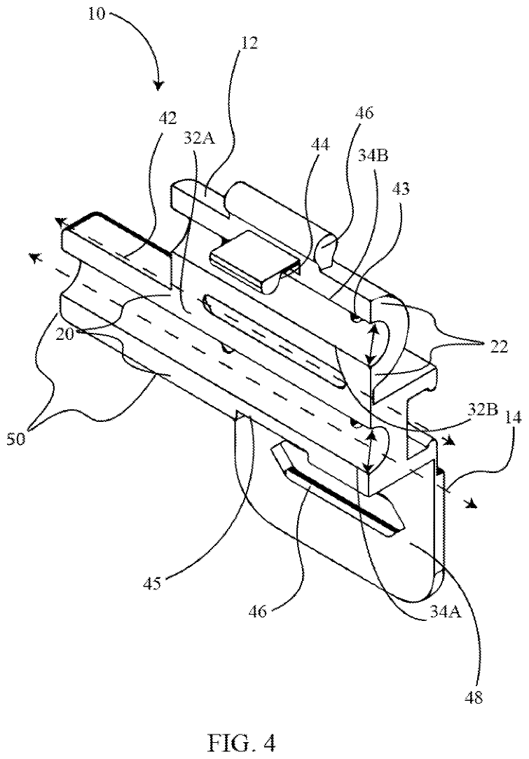

The endotracheal tube retainer 10 of the present disclosure includes a clip 12 intended to be supported on a head of a patient such that the clip is spaced apart and in front of a mouth of the patient. The clip 12 of the endotracheal tube retainer 10 defines a longitudinal axis 14 and is configured to be repeatably adjustable between an open configuration, as best shown in FIG. 4, and a closed configuration, as best shown in FIG. 2. As shown, in the closed configuration, the clip 12 defines a tube retention channel 16 configured to receive the endotracheal tube. Specifically, the clip 12 is configured to grip a longitudinal segment of the endotracheal tube in the closed configuration such that the endotracheal tube is retained in a substantially fixed relation with respect to the clip. Further, in the illustrated embodiment, the clip 12 retains the endotracheal tube in the tube retention channel 16 such that the tube extends longitudinally along the longitudinal axis 14 of the clip. When the clip 12 is in the open configuration, it releases the endotracheal tube such that the endotracheal tube is movable relative to the clip.

In the illustrated embodiment, shown in FIG. 3, the clip 12 comprises a lower half-tube portion 20 (broadly, a first portion) supported on the platform coupling 18 and an upper half-tube portion 22 (broadly, a second portion) that is movably connected to the lower half-tube portion for opening and closing the clip. The lower half-tube portion and the upper half-tube portion are configured to be brought together to form a generally tube-shaped body 26. The tube-shaped body 26 has an interior surface (e.g., a cylindrical interior surface) that defines the retention channel 16. As one would understand, an inner diameter 28 (broadly, inner cross-sectional dimension) of the generally tube-shaped body 26 can be of varying size such that endotracheal tubes of varying outer diameters can be retained in correspondingly sized clips 12. In the current embodiment, as shown in FIGS. 2 and 3, the lower and upper half-tube portions 20, 22 each define about 180 degrees—in other words, roughly equal amounts—of a cylindrical inner perimeter surface of the generally tube-shaped body 26, which defines the retention channel 16. However, it will be understood that “half-tube portions” could be of unequal size without departing from the scope of the disclosure. Further, a two-part tube-shaped body 26 is not a necessary feature of every clip within the scope of the disclosure. Other types of clips (e.g., strap-type clips, spring clips) that can open and close around an endotracheal tube may also be used without departing from the scope of the disclosure.

Referring to FIGS. 2 and 3, the upper half-tube portion 22 is movable with respect to the lower half-tube portion 20 between an open position and a closed position to adjust the clip from the open configuration in FIG. 4 to the closed configuration in FIG. 3. As shown in FIG. 4, each of the lower half-tube portion 20 and the upper half-tube portion 22 includes a respective first longitudinal edge margin 32A, 32B (which may be referred to as a “hinged edge margin”) and a respective second longitudinal edge margin 34A, 34B (which may be referred to as a “non-hinged edge margin”). As shown in FIG. 3, when the upper half-tube portion 22 is in the closed position, the hinged longitudinal edge margin 32A of the lower half-tube portion 20 and the hinged longitudinal edge margin 32B of the upper half-tube portion engage one another at a first (hinged) longitudinal edge interface 36. Likewise, the non-hinged longitudinal edge margin 34A of the lower half-tube portion and the non-hinged longitudinal edge margin 34B of the upper half-tube portion engage one another at a second (non-hinged) longitudinal edge interface 38. As shown, the tube retention channel 16 is spaced apart between the first and second longitudinal edge interfaces 36, 38.

The illustrated clip 12 further includes a hinge portion 40 (e.g., a living hinge). The hinge portion 40 connects the hinged longitudinal edge margin 32A of the lower half-tube portion 20 and the hinged longitudinal edge margin 32B of the upper half-tube portion 22 and defines a hinge axis 42 (which is parallel to the longitudinal axis 14 in the illustrated embodiment). The upper half-tube portion 22 is generally rotatable about the hinge axis 42 with respect to the lower half-tube portion 20 between the open position and the closed position.

Further, as shown in FIG. 4, the lower half-tube portion 20 and the upper half-tube portion 22 of the clip 12 can additionally include one or more gripping protrusions 43 on the generally cylindrical inner surface 30. In one or more embodiments, each gripping protrusion 43 comprises a generally dome-shaped feature that protrudes radially inward with respect to the longitudinal axis 14 from the cylindrical inner surface 30. When the clip 12 closes around an endotracheal tube, the protrusions 43 press radially inwardly on the tube to provide additional grip. In a further embodiment, the clip 12 may be formed from additional material (e.g., silicon) along the tube retention channel 16 such that the additional material can provide an increased grip.

As best shown in FIG. 4, the clip 12 of the endotracheal tube retainer 10 includes a first fastener for releasably retaining the clip in the closed configuration and also a second fastener for releasably retaining the clip in the closed configuration independently of the first fastener. In other words, the illustrated clip 12 comprises primary and secondary fasteners for independently retaining the clip in the closed configuration. In the illustrated embodiment, the primary fastener comprises latch elements 44, 45 configured to latch together to releasably retain the lower half-tube portion 20 and upper half-tube portion 22 in the closed position. More particularly, the primary fastener comprises an upper latch arm 44 depending from the non-hinged edge margin 34B of the upper half-tube portion 22 and a corresponding latch recess 45 on the non-hinged side of the lower half-tube portion 20. The latch arm 44 is configured to snap into the latch recess 45 as the upper half-tube portion 22 of the clip 12 moves from the open position to the closed position. This secures the primary fastener for retaining the clip 12 in the closed configuration. It can be seen that the primary fastener is configured to be closed by a one-handed operation. For instance, the primary fastener can be closed by simply squeezing the upper and lower half-tube portions 20, 22 together using only one hand, causing the latch arm 44 to automatically latch with the latch recess 45. The primary fastener can also be released with one hand (one-handed operation) by bending the latch arm 44 outward in a direction opposite the hinge 40 to dislodge it from the latch recess 45. When the primary fastener is released, the user can freely move the upper half-tube portion 22 to the open position (e.g., using the same hand used to bend the latch arm 44 out of the recess 45).

In the illustrated embodiment, the secondary fastener comprises a snap arm 46 (broadly, a secondary retention feature) extending outward from the non-hinged side of the upper half-tube portion 22 and a retention flap 48 bendably connected to the lower portion of the retainer 12 near the platform coupling 18. The retention flap 48 is configured to be selectively engaged with the snap arm 46 when the upper half-tube portion 22 is in the closed position such that the retention flap can further retain the upper half-tube portion in the closed position. In the illustrated embodiment, the snap arm 46 is elongate along the longitudinal axis 14. In cross-section, the snap arm comprises an enlarged outer head portion and a narrower inner neck portion that holds the head portion on the half-tube portion 22. The retention flap 48 comprises an elongate slot that is elongated along the longitudinal axis 14. When the clip 12 is in the closed configuration—which automatically latches the primary fastener to hold the clip closed as explained above—one hand can bend the retention flap 48 upward and press-fit the flap onto the snap arm 46. The head portion of the snap arm 46 snaps through the slot in the retention flap 48 and then holds the retention flap in place at the neck portion of the snap arm. It can be seen that, in this position, the retention flap 48 and the snap arm 46 resist any forces tending to open the clip 12. To open the clip 12, a user must first bend the retention flap 48 downward to separate it from the snap arm 46. In the illustrated embodiment, the relatively large retention flap 48 can easily be released using only one hand. In a non-illustrated embodiment, the retention flap 48 can alternatively be a flexible material such that the retention flap can be bent generally downwards to wrap around the platform P and the clip 12, such that the retention flap press-fits the flap onto the snap arm 46 when the upper half-tube portion 22 is in the closed position. This allows for additional stability of the endotracheal tube holder 10 onto the platform P by the platform coupling 18 by providing further means of attachment of the endotracheal tube holder 10 onto the platform P.

During ordinary use, the first or primary fastener latches first and automatically when the clip is closed. The second or secondary fastener must be separately manipulated to independently secure the clip closed. It can be seen that the primary and secondary clips provide redundant retention of the clip 12 in the closed configuration. This helps ensure that a patient's (e.g., a neonatal patient's) incidental movements do not inadvertently release the endotracheal tube from the retainer 10 by accidental opening of the clip 12. It will be understood that an endotracheal tube retainer could have other configurations of redundant primary and secondary fasteners without departing from the scope of the disclosure.

As shown in FIGS. 2 and 4, in the preferred embodiment, the lower portion 20 of the endotracheal tube retainer 10 comprises a stabilizer extension 50 that protrudes from the clip 12 along the longitudinal axis 14 of the clip. It can be seen that, when an endotracheal tube is retained in the retainer 10, a first longitudinal segment of the tube will be received in the clip 12 and a second longitudinal segment of the tube will be supported on the stabilizer extension 50. In the preferred embodiment, the stabilizer extension 50 is configured to extend in a side-by-side relation with lower portion (broadly, a first perimeter portion) of the second longitudinal segment of the endotracheal tube and to expose an upper portion (broadly, a second perimeter portion diametrically opposite the first perimeter portion) of the second longitudinal segment.

The stabilizer extension 50 is configured to support and stabilize the endotracheal tube such that a user (e.g., as a physician or physician aid) can use one hand to stabilize the endotracheal tube on the retainer 10, even when the clip 12 is not closed. More particularly, the user positions one or more fingers below the stabilizer extension 50 and one or more fingers above the endotracheal tube and presses the endotracheal tube against the stabilizer extension. This provides a firm one-handed grip that enables the user to securely hold the endotracheal tube in place in relation to retainer 10. Further it allows the user to use the single tube-gripping hand to selectively release some of the gripping force as desired to allow for controlled movement or adjustment of the endotracheal tube, e.g., for slight adjustments of the endotracheal tube in relation to the patient.

To use the tube retainer 10, it first must be placed on an endotracheal tube holder H. After a patient is intubated, the tube holder can be positioned on the patient's head in the known manner But instead of securing the tube to the platform P using tape, the user closes the clip 12 around the tube, thereby fixing the tube in place in relation to the holder H.

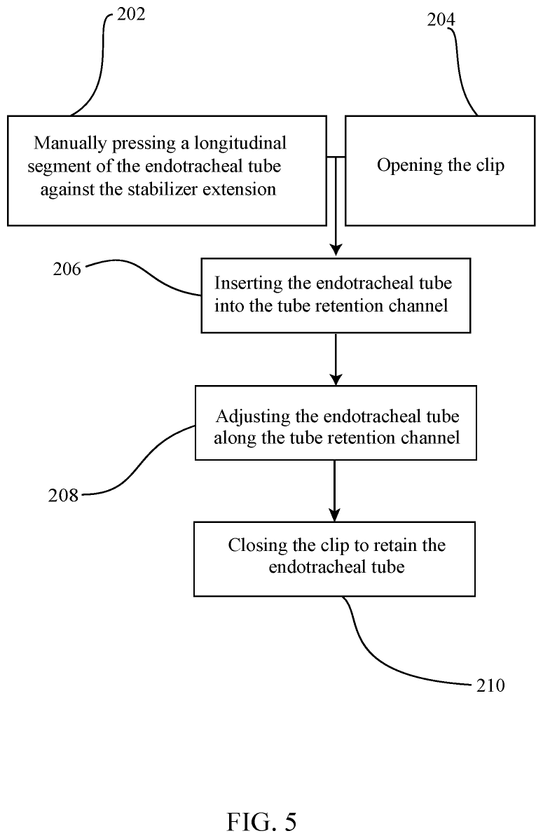

Referring to FIG. 5, the illustrated retainer 10 enables a method of adjusting the position of the endotracheal tube 200 that can be reliably performed by a single person after the endotracheal tube is initially secured in the retainer. At step 202, the user manually presses a longitudinal segment of the endotracheal tube against the stabilizer extension 50 to stabilize the endotracheal tube against the stabilizer extension in the manner described above. In an exemplary embodiment, the user performs step 202 using only a first hand. While performing step 202, using only the user's second hand, the user releases the clip 12 in the manner described above. First the user releases the retention flap 48 from the snap arm 46, and then the user releases the latch hook 44 from the latch recess 45. This releases the clip so that, using the same second hand, the user can move the upper half-tube portion 22 to the open position. In one or more embodiments, the hinge portion 40 is configured to allow the clip to remain open, e.g., the hinge portions are not strongly resilient such that they tend to always force the clip closed in the manner of a spring clip. At step 206, the endotracheal tube, already intubated, can be inserted into the tube retention channel 16. Thus, when the clip 12 is open, the user can use the second hand to adjust the endotracheal tube position (step 208). However, adjusting of the endotracheal tube position can be done before or after any one of the plurality of steps in method 200. During this step the user's first and second hands work in coordination. The user's first hand adjusts the gripping force applied to the endotracheal tube as needed to allow the second hand to move the tube to the desired position. However, in an exemplary embodiment, the user maintains a somewhat firm grip on the tube and the stabilizer extension at all times. This helps ensure that no sudden, unexpected movements occur that might cause inadvertent extubation. When the endotracheal tube has been adjusted to the desired position, the user can close the clip 12 with the second hand while holding the tube in place using the first hand (step 210). With the user's first hand, the user moves the upper half-tube portion 22 to the closed position, which automatically latches the latch hook 44 with the latch recess 45 and thereby provides primary retention of the tube in the tube retainer. Subsequently, the user bends the retention flap 48 up until it snaps onto the snap arm 46, providing secondary retention of the clip 12 in the closed position.

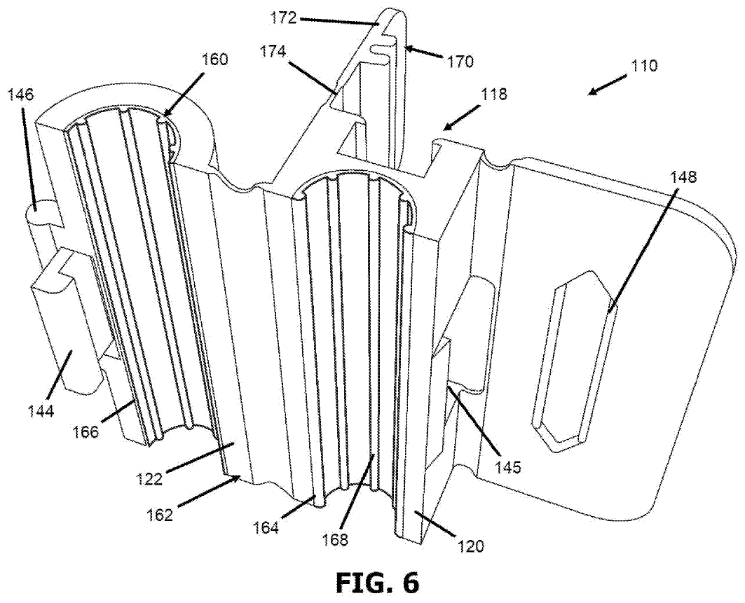

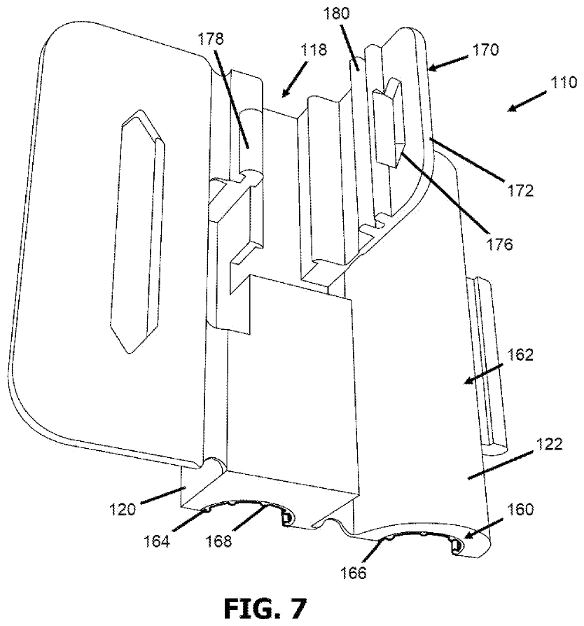

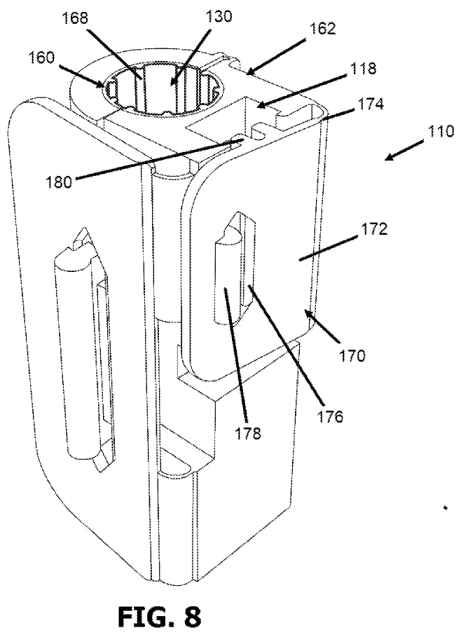

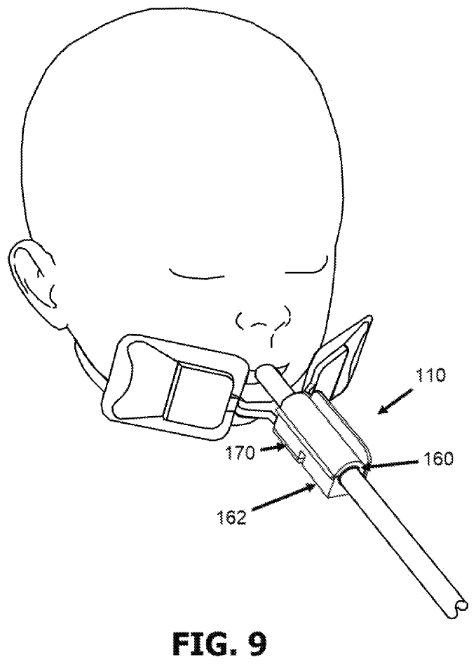

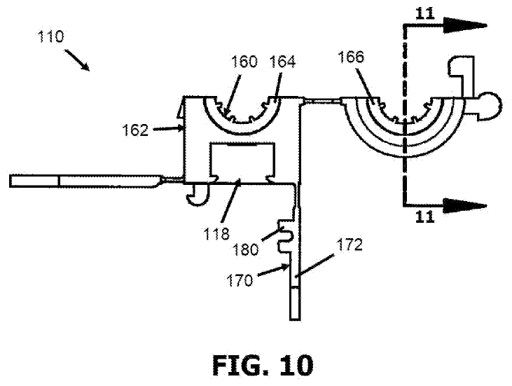

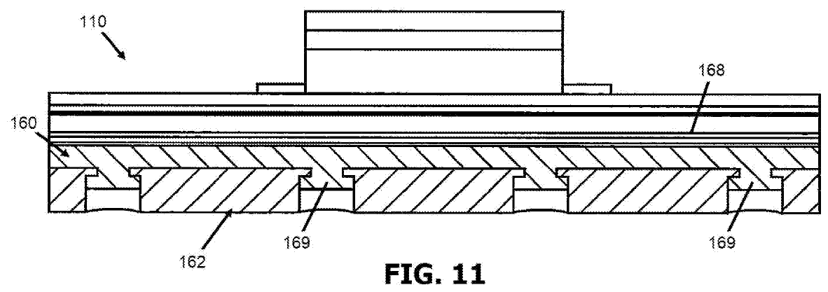

Referring to FIGS. 6-11, another embodiment of an endotracheal tube retainer is generally indicated at reference number 110. The endotracheal tube retainer 110 is substantially similar to the endotracheal tube retainer 10 described above. The parts of the endotracheal tube retainer 110 discussed below that correspond to parts of the endotracheal tube 10 discussed above are given the same reference number, plus 100. Only the features that distinguish the endotracheal tube retainer 110 from the endotracheal tube retainer 10 are discussed below. It is to be understood that the retainer 110 can possess all other aspects of the endotracheal tube retainer 10 not expressly distinguished in the following disclosure. As explained below, the endotracheal tube retainer 110 differs from the endotracheal tube retainer 10 in three respects. First, the stabilizer extension 50 is omitted. Second, an overmolded tube contact element 160 is included in the clip 112. Third, a secondary platform coupling holder 170 is provided for redundant retention of a tube holder platform P in the platform coupling 118.

In the illustrated embodiment, the endotracheal tube retainer 10 is formed from two parts, a tube contact element 160 defining the tube retention channel 130 and an overmold 162 formed on the tube contact element. Suitably, the tube contact element 160 and the overmold 162 are formed of different materials. For instance, the overmold is formed from a more rigid material than the tube contact element. In an exemplary embodiment, the overmold 162 comprises rigid polypropylene and the tube contact element 160 comprises soft TPE material. The softer tube contact element 160 forms the point of contact with the endotracheal tube during use. It is believed that providing a softer point of contact with the tube may mitigate against kinking the tube in the clip 112.

In the illustrated embodiment, the tube contact element 160 comprises a first half-tube contact element 164 received in the lower half-tube portion 120 of the clip 112 and a second half-tube contact element 166 received in the upper half-tube portion 122 of the clip. When the clip 112 is closed, the first and second half-tube contact elements 164 a 360° cylinder configured to grip the endotracheal tube of corresponding size. The tube contact element 160 comprises a plurality of longitudinal gripping ribs 168 (each, broadly, a gripping protrusion) for gripping the endotracheal tube in the tube retention channel 130. Each half-tube contact element 164, 166 further comprises plurality of external protrusions 169 for interlocking with the overmold 162 once the overmold is overmolded on the tube contact element 160.

In the illustrated embodiment, the overmold 162 forms all other in-use features of the endotracheal tube retainer 110. That is, the overmold 162 forms the primary fastener elements 144, 145 for releasably retaining the clip 112 in the closed configuration, the secondary fastening elements 146, 148 for releasably retaining the clip 112 in the closed configuration, the platform coupling 118, and the secondary platform holder 170 discussed in further detail below.

In addition to providing a soft point of contact with the endotracheal tube, forming the retainer 110 from a tube contact element 160 and an overmold 162 is believed to provide additional benefits for manufacturing. For example, the endotracheal tube retainer 110 can be formed in a variety of tube sizes using only a single mold tool for the complex external geometry of the device. That is, a plurality of tube contact elements 160 of different internal dimensions can be overmolded in the same mold tool to form endotracheal tube retainers 110 of different sizes using the same mold tool. It is contemplated that the same mold tool can be used to form overmolds for tube contact elements defining any inner diameter in an inclusive range extending from a 2.5 mm or less to 4.0 mm or more. For, instance, it is contemplated that the endotracheal tube retainer 110 could comprise an internal diameter of about 2.5. mm, about 3.0 mm, about 3.5 mm, or about 4.0 mm.

In an exemplary method of making an endotracheal tube retainer 110, a tube contact element 160 is provided (e.g., molded in a first mold) in a size selected for gripping an endotracheal tube of a given diameter. Subsequently, the overmold 162 is formed (e.g., molded in a second mold) on the tube contact element 160 such that the tube contact element and overmold collectively form the clip 112 and optionally the platform coupling 118.

The illustrated tube retainer 110 comprises a platform coupling 118 that has the same features as the platform coupling 18 above. In addition, the tube retainer 110 comprises the secondary platform holder 170 configured to hold the platform P in the platform coupling 118. The secondary platform holder 170 comprises a flap 172 connected to the platform coupling 118 by a living hinge 174. The secondary platform holder 170 and the platform coupling 118 comprise mutual latch features 176, 178 configured to latch together to releasably retain the secondary platform holder at a location at which it holds the platform P in the platform coupling, e.g., to retain the flap 172 so that it covers the bottom opening through which the platform snaps into the platform coupling. In the illustrated embodiment, the flap 172 comprises a latch recess 176 and the platform coupling comprises a latch hook protrusion 178 configured to latch with the latch recess. The flap 172 comprises one or more gripping protrusions 180 configured to be compressed against the platform P in the coupling 118 when the flap is latched to the platform coupling via the latch hook protrusion 178 and latch recess 176.

### Other Statements of Invention that May be Claimed

In addition to the claims set forth below, the following additional statements may also form the basis for claims:
A. The endotracheal tube retainer as set forth in any of claims 1-9, 11-19, and 23-24 below, wherein the clip includes a tube contact element defining the tube retention channel and an overmold formed on the tube contact element.
B. The endotracheal tube retainer of statement A, wherein the tube contact element and the overmold are formed of different materials.
C. The endotracheal tube retainer of either of statements A and B, wherein the overmold defines one or more fasteners configured to releasably retain the clip in the closed configuration.
D. The endotracheal tube retainer as set forth in any of statements A-C, wherein the overmold defines a platform coupling configured to fasten the endotracheal tube retainer on a separate platform of an endotracheal tube holder.
E. The endotracheal tube retainer as set forth in any of statements A-D, wherein the tube contact element comprises first and second half-tube contact elements configured to form a 360° cylinder when the clip is in the closed configuration.
F. The endotracheal tube retainer as set forth in any of statements A-E, wherein the tube contact element comprises a gripping protrusion for gripping the endotracheal tube in the tube retention channel
G. The endotracheal tube retainer as set forth in statement F, wherein the gripping protrusion comprises a plurality of longitudinal ribs.
H. The endotracheal tube retainer as set forth in any of statements A-G, wherein the tube contact element comprises a plurality of external protrusions for interlocking with the overmold.
I. A method of making an endo tracheal tube retainer, the method comprising:
providing a tube contact element of a size selected for gripping an endotracheal tube; and
forming an overmold on the tube contact element such that the tube contact element and overmold collectively form a clip corresponding to the clip recited in any of claims 1-9, 11-19, and 23-24 below or statements A-H above.
J. The endotracheal tube retainer as set forth in claim 23 below, further comprising a secondary platform holder configured to hold the platform in the platform coupling.
K. The endotracheal tube retainer as set forth in statement J, wherein the secondary platform holder comprises a flap connected to the platform coupling by a living hinge.
L. The endotracheal tube retainer as set forth in statement J and K, wherein the secondary platform holder and the platform coupling comprise mutual latch features configured to latch together to releasably retain the secondary platform holder at a location at which it holds the platform in the platform coupling.

The foregoing description has been presented for the purposes of illustration and description. It is not intended to be exhaustive or to limit the disclosure to the precise form disclosed. Many modifications and variations are possible in view of this disclosure. Indeed, while certain features of this disclosure have been shown, described and/or claimed, it is not intended to be limited to the details above, since it will be understood that various omissions, modifications, substitutions and changes in the apparatuses, forms, method, steps and system illustrated and, in its operation, can be made by those skilled in the art without departing in any way from the spirit of the present disclosure.

Furthermore, the foregoing description, for purposes of explanation, used specific nomenclature to provide a thorough understanding of the disclosure. However, it will be apparent to one skilled in the art that the specific details are not required in order to practice the disclosure. Thus, the foregoing descriptions of specific embodiments of the present disclosure are presented for purposes of illustration and description. They are not intended to be exhaustive or to limit the disclosure to the precise forms disclosed, many modifications and variations are possible in view of the above teachings. The embodiments were chosen and described in order to best explain the principles of the disclosure and its practical applications, to thereby enable others skilled in the art to best utilize the disclosed system and method, and various embodiments with various modifications as are suited to the particular use contemplated.

1. An endotracheal tube retainer for retaining an endotracheal tube in an intubated position on a subject, the endotracheal tube retainer comprising a clip configured to be supported on a head of a subject such that the clip is spaced apart in front of a mouth of the subject, the clip being repeatably adjustable between an open configuration and a closed configuration, the clip in the closed configuration defining a tube retention channel, the clip in the closed configuration being configured to receive the endotracheal tube in the tube retention channel such that the clip grips a longitudinal segment of the endotracheal tube to retain the endotracheal tube in substantially fixed relation with respect to the clip, the clip in the open configuration being configured to release the endotracheal tube such that the endotracheal tube is movable relative to the clip.

2. The endotracheal tube retainer of claim 1, wherein the clip is adjustable from the closed configuration to the open configuration by a one-handed operation.

3. The endotracheal tube retainer of claim 1, wherein the clip is adjustable from the open configuration to the closed configuration by a one-handed operation.

4. The endotracheal tube retainer of claim 1, wherein the clip comprises a fastener configured to releasably retain the clip in the closed configuration, the fastener being releasable by a one-handed operation.

5. The endotracheal tube retainer of claim 1, wherein the clip has a longitudinal axis, the clip being configured to retain the endotracheal tube in the tube retention channel such that the tube extends longitudinally through the tube retention channel generally along the longitudinal axis.

6. The endotracheal tube retainer of claim 5, further comprising a stabilizer extension extending from the clip along the longitudinal axis.

7. The endotracheal tube retainer of claim 6, wherein said longitudinal segment of the endotracheal tube is a first longitudinal segment, wherein the stabilizer extension is configured to extend along a second longitudinal segment of the endotracheal tube spaced apart from the first longitudinal segment when the clip grips the first longitudinal segment of the endotracheal tube.

8. The endotracheal tube retainer of claim 7, wherein the stabilizer extension is configured to extend in side-by-side relation with a first perimeter portion of the second longitudinal segment of the endotracheal tube and to expose a second perimeter portion of the second longitudinal segment diametrically opposite the first perimeter portion.

9. The endotracheal tube retainer of claim 8, wherein the stabilizer extension is configured to support and stabilize the endotracheal tube such that a user can manually press the second perimeter portion of the second longitudinal segment toward the stabilizer extension to hold the second longitudinal segment against the stabilizer extension.

10. A method of adjusting an intubated endotracheal tube retained by the endotracheal tube retainer of claim 9, the method comprising:
manually pressing a longitudinal segment of the endotracheal tube against the stabilizer extension to stabilize the endotracheal tube against the stabilizer extension;
while manually pressing the longitudinal segment of the endotracheal tube against the stabilizer extension, opening the clip;
after opening the clip, adjusting the endotracheal tube; and
after adjusting the endotracheal tube, closing the clip to retain the endotracheal tube in an adjusted position.

11. The method of claim 9, wherein said manually pressing is conducted with only an individual's first hand and at least one of said opening and said closing is conducted with only the individual's second hand.

12. The endotracheal tube retainer of claim 1, wherein the clip comprises a first portion and a second portion movable with respect to the first portion between an open position and a closed position to adjust the clip from the open configuration to the closed configuration.

13. The endotracheal tube retainer of claim 12, wherein each of the first portion and the second portion comprises a first longitudinal edge margin and a second longitudinal edge margin, wherein when the second portion is in the closed position, the first longitudinal edge margins of the first and second portions engage one another at a first longitudinal edge interface and the second longitudinal edge margins of the first and second portions engage one another at a second longitudinal edge interface, the tube retention channel being spaced apart between the first and second longitudinal edge interfaces.

14. The endotracheal tube retainer of claim 13, wherein the clip comprises a hinge portion connecting the first longitudinal edge margins of the first and second portions and defining a hinge axis, the second portion being generally rotatable about the hinge axis with respect to the first portion between the open position and the closed position.

15. The endotracheal tube retainer of claim 13, wherein the first and second portions of the clip comprise mutual latch elements configured to latch together to releasably retain the second portion in the closed position.

16. The endotracheal tube retainer of claim 15, wherein the mutual latch elements are configured to snap together as the second portion of the clip moves from the open position to the closed position.

17. The endotracheal tube retainer of claim 15, wherein the second portion of the clip further comprises a secondary retention feature and the clip further comprises a retention flap hingedly connected to the first portion of the clip and configured to be selectively engaged with the secondary retention feature when the second portion is in the closed position such that the retention flap holds the second portion in the closed position.

18. The endotracheal tube retainer as set forth in claim 12, wherein each of the first portion comprises a first half-tube portion and the second portion comprises a second half-tube portion, and in the closed configuration of the clip the first and second half-tube portions are brought together to form a generally tube-shaped body.

19. The endotracheal tube retainer as set forth in claim 18, wherein the generally tube-shaped body comprises a generally cylindrical inner surface defining the tube retention channel and one or more gripping protrusions formed on the generally cylindrical inner surface.

20. An endotracheal tube holder comprising:
a support bar having a length and a middle portion along the length, the support bar being configured to be attached to a head of a subject such that the support bar extends lengthwise in a generally ear-to-ear direction and the middle portion of the support bar is spaced apart in front of the mouth;
a platform extending transversely from the middle portion of the support bar; and
the endotracheal tube retainer as set forth in claim 1 supported on the platform.

21. The endotracheal tube holder as set forth in claim 20, wherein the endotracheal tube retainer and the platform are integrally formed from a single piece of monolithic material.

22. The endotracheal tube holder as set forth in claim 20, wherein the endotracheal tube retainer is separately attached to the platform.

23. The endotracheal tube retainer of claim 1, further comprising a platform coupling for attaching the endotracheal tube retainer to a platform of a separate endotracheal tube holder.

24. The endotracheal tube retainer of claim 1, wherein the clip comprises a first fastener for releasably retaining the clip in the closed configuration and a second fastener for releasably retaining the clip in the closed configuration independently of the first fastener.
